(19) United States
(12) Patent Application Publication
(10) Pub. No.: US 2023/0206097 A1
(43) Pub. Date: Jun. 29, 2023

(54) THOUGHT INFERENCE SYSTEM, INFERENCE MODEL GENERATION SYSTEM, THOUGHT INFERENCE DEVICE, INFERENCE MODEL GENERATION METHOD, AND NON-TRANSITORY COMPUTER READABLE STORAGE MEDIUM

(71) Applicant: National University Corporation Ehime University, Matsuyama-shi (JP)

(72) Inventor: Tomonori KARITA, Matsuyama-shi (JP)

(21) Appl. No.: 18/178,922

(22) Filed: Mar. 6, 2023

Related U.S. Application Data
(63) Continuation of application No. PCT/JP2021/034867, filed on Sep. 22, 2021.

(30) Foreign Application Priority Data
Sep. 23, 2020 (JP) 2020-158438

(51) Int. Cl. G06N 5/045; G16H 40/67

(52) U.S. Cl. CPC G06N 5/045; G16H 40/67

(57) ABSTRACT

A care support server acquires a plurality of data sets each of which indicates a first condition for a case where a first physical reaction is seen in a person with speech difficulties and a first thought of the person with speech difficulties for a case where the first physical reaction is seen, and generates an inference model by machine learning in which the first condition indicated in each of the plurality of data sets is used as an explanatory variable and the first thought indicated in each of the plurality of data sets is used as an objective variable. The care support server then inputs input data indicating a second condition for a case where a second reaction is seen to the inference model to infer a second thought for the case where the second reaction is seen, and outputs the inference result.

### FIELD

The present invention relates to a technique for inferring thoughts such as desires of people with speech difficulties.

### BACKGROUND

Electrical appliances such as air conditioners, televisions, lighting equipment, and microwave ovens controlled with a user's voice have attained widespread use. Electrical appliances that can respond to only very limited commands were already in widespread use at the end of the 20^{th} century. Recent years haves seen the use of electrical appliances controllable with a user's voice more humanly or interactively using technologies of artificial intelligence (AI), Internet of things (IoT), and information and communications technology (ICT). Electrical appliances controllable with a voice using the technologies are called “smart home appliances”, “IoT home appliances”, “AI home appliances”, or the like.

Further, smart speakers use the technologies of AI, IoT, and ICT to search for information and present the information found out by the search or to control electrical appliances based on a user's voice. Such functions are available also on mobile devices of smart phones, tablet computers, smart watches, and so on.

Hereinafter, such smart home appliances, smart speakers, and mobile devices are referred to as “ICT devices”.

As described above, a user can use an ICT device more humanly or interactively. Accordingly, also for people with physical disabilities, the ICT device is easier to use than conventional devices, so that the quality of life (QOL) of people with physical disabilities can be improved.

However, it is still difficult for the ICT device to directly improve the QOL of people with speech difficulties, e.g., people with severe motor and intellectual disabilities or dementia patients. This is because the voice of people with speech difficulties is still difficult to be recognized by the speech recognition function employed in the ICT device. In view of this, the ICT device has been used through a caregiver such as a family member or a healthcare worker.

### CITATION LIST

### Patent Literature

Patent Literature 1: Japanese Patent No. 6315744

### SUMMARY

### Technical Problem

In the meantime, a conversation assistance terminal described in Patent Literature 1 includes an input device and a display device. In a case where an image displayed in the display device is selected, the conversation assistance terminal outputs, by voice, a message corresponding to the selected image. A user of the conversation assistance terminal can select a displayed image to cause the terminal to utter a message on behalf of the user. This can cause an ICT device to hear the voice.

However, in a conventional device such as the conversation assistance terminal, it is necessary to select a displayed image; therefore, such a conventional device is sometimes difficult for a person with severe motor and intellectual disabilities or a dementia patient to use without assistance of a caregiver. In other words, such a conventional device places a burden on the caregiver.

The present invention has been achieved in light of such a problem, and therefore, an object of the present invention is to reduce the burden on the caregiver in a case where a person with severe motor and intellectual disabilities or a dementia patient uses the ICT device.

### Solution to Problem

A thought inference system according to an aspect of the present invention includes a data set acquisition module configured to acquire a plurality of data sets, each of the data sets indicating a first condition for a case where a first physical reaction is seen in a person with speech difficulties and a first thought of the person with speech difficulties for a case where the first physical reaction is seen; an inference model generation module configured to generate, for each of a plurality of combinations of a time frame and a location, an inference model by machine learning in which the first condition indicated in the data set acquired in a time frame and a location of the subject combination is used as an explanatory variable and the first thought indicated in the data set is used as an objective variable; an inference module configured to infer a second thought for a case where a second reaction is seen in the person with speech difficulties by inputting input data indicating a second condition for a case where the second reaction is seen to the inference model that is generated, among the plurality of combinations, for a combination of a time frame and a location in which the second reaction is seen; and an output module configured to output the second thought.

### Advantageous Effects of Invention

The present invention makes it possible to reduce the burden on the caregiver in a case where a person with severe motor and intellectual disabilities or a dementia patient uses the ICT device.

### BRIEF DESCRIPTION OF DRAWINGS

FIG. 1 is a diagram illustrating an example of the overall configuration of a thought inference system.

FIG. 2 is a diagram illustrating an example of the hardware configuration of a care support server.

FIG. 3 is a diagram illustrating an example of the functional configuration of a care support server.

FIG. 4 is a diagram illustrating an example of the hardware configuration of a caregiver terminal.

FIG. 5 is a diagram illustrating an example of the functional configuration of a caregiver terminal.

FIG. 6 is a diagram illustrating an example of a thought input screen.

FIG. 7 is a diagram illustrating an example of an option list.

FIG. 8 is a diagram illustrating an example of raw data.

FIG. 9 is a diagram illustrating an example of a data set.

FIGS. 10A and 10B are diagrams illustrating an example of category tables.

FIG. 11 is a diagram illustrating an example of the flow of processing for testing an inference model.

FIG. 12 is a diagram illustrating an example of raw data.

FIG. 13 is a diagram illustrating an example of the flow of inference processing.

FIG. 14 is a diagram illustrating an example of an inference result screen.

FIG. 15 is a flowchart depicting an example of the flow of the entire processing by an inference service program.

FIG. 16 is a diagram illustrating a modification to the functional configuration of the care support server.

FIG. 17 is a diagram illustrating a modification to the functional configuration of the caregiver terminal.

FIG. 18 is a diagram illustrating an example of an event table.

FIG. 19 is a diagram illustrating a modification to the option list.

FIG. 20 is a diagram illustrating an example of an explanatory variable table.

### DESCRIPTION OF EMBODIMENTS

### (Overall Configuration)

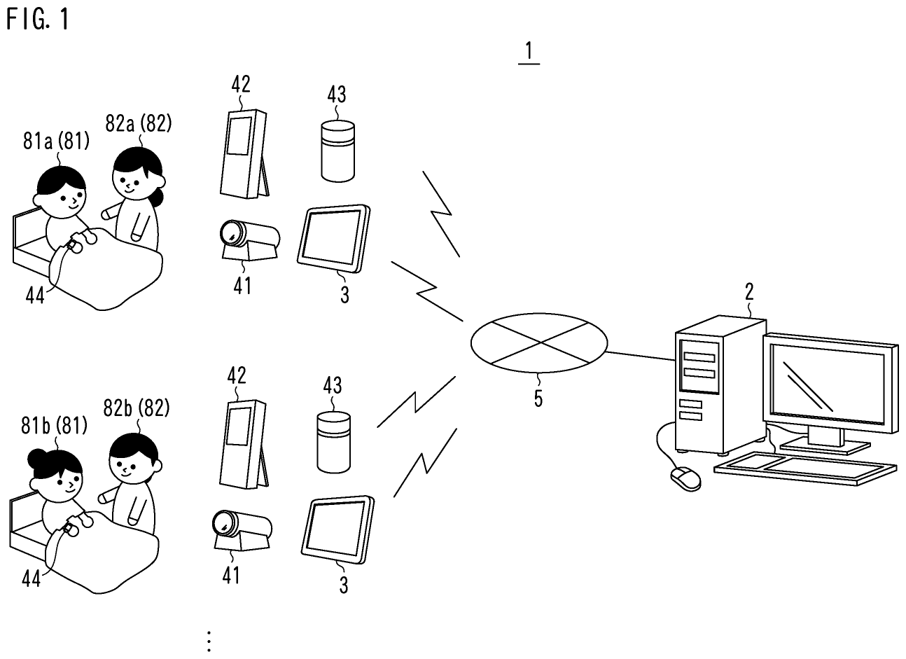

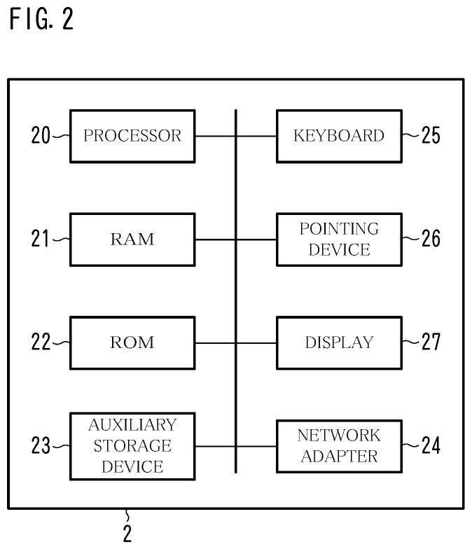

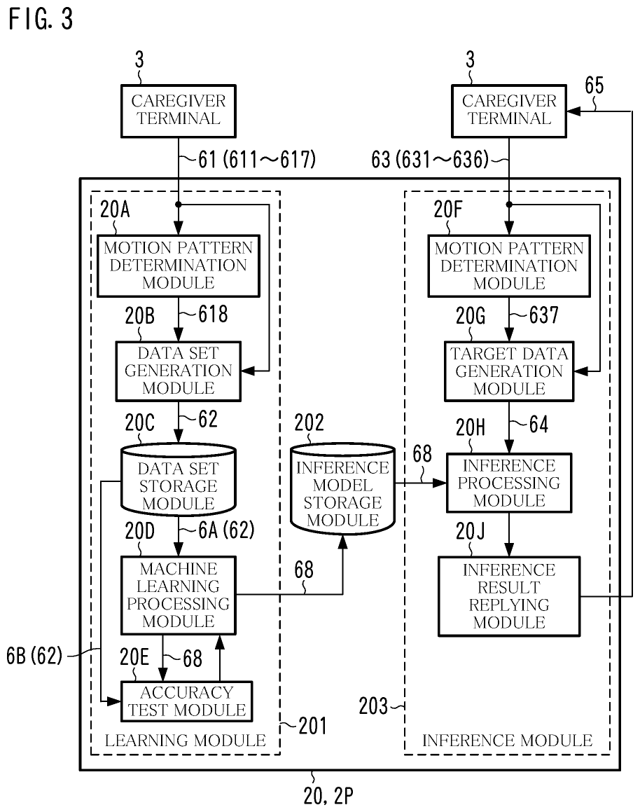

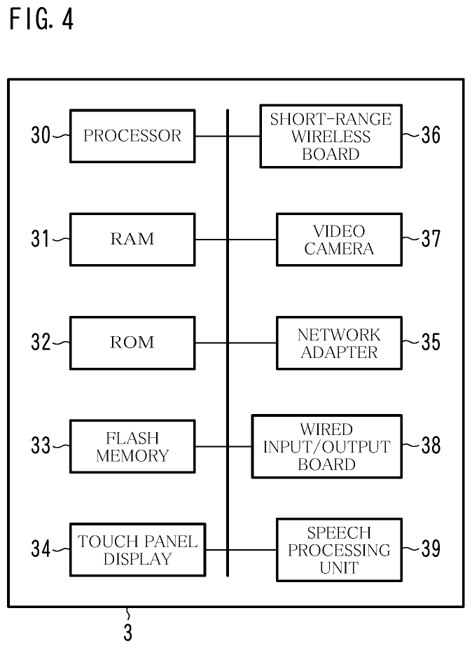

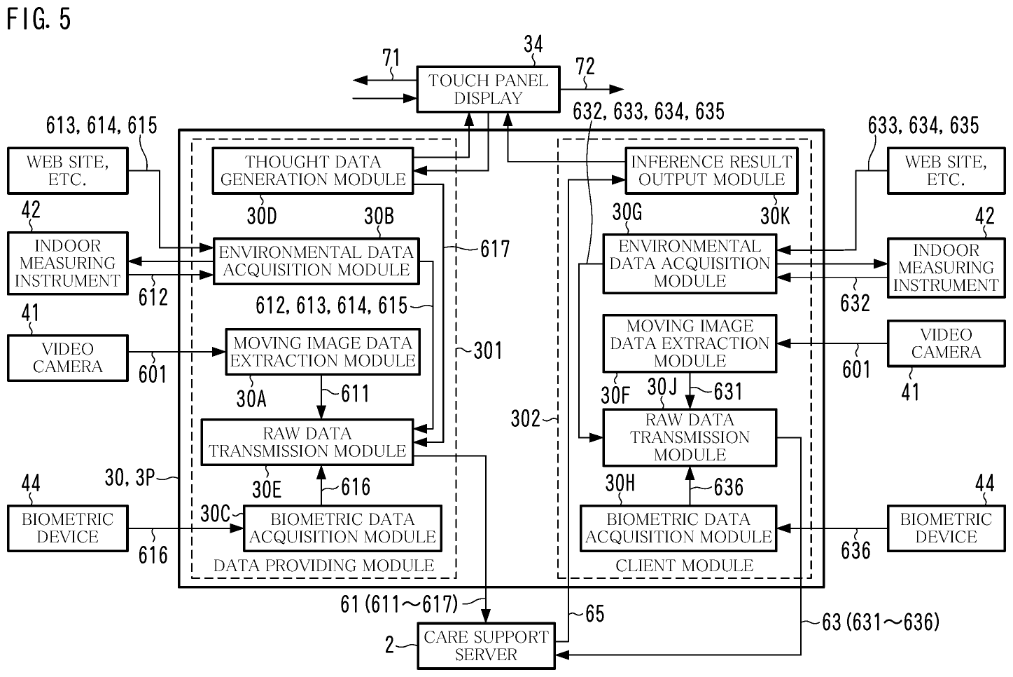

FIG. 1 is a diagram illustrating an example of the overall configuration of a thought inference system 1. FIG. 2 is a diagram illustrating an example of the hardware configuration of a care support server 2. FIG. 3 is a diagram illustrating an example of the functional configuration of the care support server 2. FIG. 4 is a diagram illustrating an example of the hardware configuration of a caregiver terminal 3. FIG. 5 is a diagram illustrating an example of the functional configuration of the caregiver terminal 3.

The thought inference system 1 illustrated in FIG. 1 is a system that infers, by artificial intelligence (AI), thoughts or ideas of a care recipient 81 to teach a caregiver 82 about the thoughts and ideas. For example, the thought inference system 1 infers and teaches a desire to “go to school” or “watch television”, a reply to a question to the care recipient 81 such as “yes”, “no”, “here”, “there”, or “I don't know”, a feeling such as “excited”, “angry”, “surprised”, or “sad”, a call such as “Did you see that, teacher?”, “Hurry!”, or “hello”, a taste such as “like” or “dislike”, a physical condition such as “sleepy”, “hungry”, “tired”, or “headache”, a sense such as “hot”, “cold”, “stinky”, “delicious”, “dazzling”, or “sour”, and other various thoughts or ideas. Hereinafter, such thoughts or ideas are referred to as “thoughts”. The caregiver 82 is, for example, a family member or a teacher of the care recipient 81, an experienced supporter, a qualified caregiver, or a healthcare worker.

The thought inference system 1 includes a care support server 2, a plurality of caregiver terminals 3, a plurality of video cameras 41, a plurality of indoor measuring instruments 42, a plurality of smart speakers 43, a plurality of biometric devices 44, and a communication line 5.

The care support server 2 and the individual caregiver terminals 3 can exchange data via the communication line 5. Examples of the communication line 5 include the Internet and a public line.

The care support server 2 generates an inference model by machine learning and infers a thought of the care recipient 81 based on the inference model. Examples of the care support server 2 include a personal computer, a workstation, and a cloud server. The following describes an example in which the care support server 2 is a personal computer.

As illustrated in FIG. 2, the care support server 2 includes a processor 20, a random-access memory (RAM) 21, a read only memory (ROM) 22, an auxiliary storage device 23, a network adapter 24, a keyboard 25, a pointing device 26, and a display 27.

The ROM 22 or the auxiliary storage device 23 stores, therein, an operating system and computer programs such as an inference service program 2P. The inference service program 2P is a program for implementing the functions of a learning module 201, an inference model storage module 202, an inference module 203, and so on, which are illustrated in FIG. 3. Examples of the auxiliary storage device 23 include a hard disk and a solid-state drive (SSD).

The RAM 21 is a main memory of the care support server 2. Computer programs such as the inference service program 2P are appropriately loaded into the RAM 21.

The processor 20 executes the computer programs loaded into the RAM 21. Examples of the processor 20 include a graphics processing unit (GPU) and a central processing unit (CPU).

The network adapter 24 performs communication with a device, e.g., the caregiver terminal 3 or a web server, using protocols such as transmission control protocol/internet protocol (TCP/IP). As the network adapter 24, a network interface card (NIC) or a communication device for Wi-Fi is used.

The keyboard 25 and the pointing device 26 are input devices for an operator to input commands or data.

The display 27 serves to display a screen with which to input commands or data, a screen showing the result of calculations by the processor 20, or the like.

Referring back to FIG. 1, the caregiver terminal 3 is used to provide the care support server 2 with data for machine learning, or to make a request for inference to receive an inference result from the care support server 2. Examples of the caregiver terminal 3 include a tablet computer, a smart phone, and a personal computer. One caregiver terminal 3 is desirably given to each of the caregivers 82, but one caregiver terminal 3 may be shared with the caregivers 82. The following description takes an example of the case where the caregiver terminal 3 is a tablet computer and one caregiver terminal 3 is given to each of the caregivers 82.

As illustrated in FIG. 4, the caregiver terminal 3 includes a processor 30, a RAM 31, a ROM 32, a flash memory 33, a touch panel display 34, a network adapter 35, a short-range wireless board 36, a video camera 37, a wired input/output board 38, and a speech processing module 39.

The ROM 32 or the flash memory 33 stores, therein, an operating system and computer programs such as a client program 3P. The client program 3P is a program for implementing the functions of a data providing module 301, a client module 302, and so on illustrated in FIG. 5. The client program 3P is downloaded from an application server on the Internet to the caregiver terminal 3 and is installed into the flash memory 33 by an installer. The installer may be downloaded from the application server together with the client program 3P, or prepared in the ROM 32 or the flash memory 33 in advance.

The RAM 31 is a main memory of the caregiver terminal 3. Computer programs such as the client program 3P are appropriately loaded into the RAM 31. The processor 30 executes the computer programs loaded into the RAM 31.

The touch panel display 34 includes a touch panel for the caregiver 82 to input commands or data and a display on which to display a screen.

The network adapter 35 performs communication with another device, e.g., the care support server 2, using protocols such as TCP/IP. Examples of the network adapter 35 include a communication device for Wi-Fi.

The short-range wireless board 36 performs communication with a device within a few meters from the caregiver terminal 3. In particular, in the present embodiment, the short-range wireless board 36 performs communication with the video camera 41, the indoor measuring instrument 42, and the biometric device 44. Examples of the short-range wireless board 36 include a communication device for Bluetooth.

The video camera 37 captures a moving image to generate moving image data. In particular, in the present embodiment, the video camera 37 is used to generate moving image data on the care recipient 81.

The wired input/output board 38 is a device performing communication with peripheral devices by wire. The wired input/output board 38 has a connection port to which the peripheral devices are connected directly or via a cable. Examples of the wired input/output board 38 include an input/output device of a universal serial bus (USB) standard.

The speech processing module 39 includes a speech processing device, a microphone, and a speaker. A sound captured by the microphone is encoded into audio data by the speech processing device, and audio data sent from another device is decoded to a sound by the speech processing device and the sound is reproduced by the speaker.

Referring back to FIG. 1, the video camera 41 is used to capture an image of the care recipient 81. The video camera 41 further includes a microphone and a speech processing device, which enables also capturing a sound to generate audio data. Examples of the video camera 41 include a small video camera, which is commercially available, having a Bluetooth, Wi-Fi, or USB communication function and a sound collection function.

In order to reduce a burden on the caregiver 82, it is desirable that one video camera 41 is given to each of the care recipients 81 and the video camera 41 is placed in advance so that the care recipient 81 is within an image-capturing range. It is of course possible that the video camera 37 of the caregiver terminal 3 is used to capture images and the microphone of the speech processing module 39 is used to pick up sounds. For example, in a case where the care recipient 81 and the caregiver 82 are out of the house, the caregiver terminal 3 may be used, instead of the video camera 41, to capture an image and pick up sounds. The following description takes an example in which the video camera 41 is used for image capturing and sound pickup.

The indoor measuring instrument 42 measures the state of a room where the care recipient 81 is present. Specifically, the indoor measuring instrument 42 measures the temperature, humidity, air pressure, illuminance, amount of ultraviolet (UV) radiation, pollen level, and the like inside the room. Examples of the indoor measuring instrument 42 include a small measuring instrument for environmental measurement, which is commercially available, having a Bluetooth, Wi-Fi, or USB communication function. A device in which a plurality of commercially available sensors is combined may be used as the indoor measuring instrument 42. For example, a device in which various sensors such as a temperature/humidity sensor, an air pressure sensor, an illuminance sensor, a UV sensor provided by ALPS ALPINE CO., LTD. are combined with one another. Incidentally, the pollen level is the amount of pollen per unit volume.

The indoor measuring instrument 42 is installed for each room where the care recipient 81 is present. In a case where the care recipients 81 are present in one room, only one indoor measuring instrument 42 may be installed in the room and shared with the care recipients 81, or, alternatively, one indoor measuring instrument 42 may be installed near each of the care recipients 81. Further, when going out, the care recipient 81 or the caregiver 82 may carry the indoor measuring instrument 42.

The smart speaker 43 recognizes a sound to perform tasks based on the recognition result, for example, to search for information or control electrical appliances. In the present embodiment, particularly, the smart speaker 43 performs the tasks for the care recipient 81. Examples of the smart speaker 43 include a commercially available smart speaker having a Bluetooth, Wi-Fi, or USB communication function.

The biometric device 44 measures biometric information such as heart rate, pulse wave, blood oxygen level, blood pressure, electrocardiographic potential, body temperature, myoelectric potential, and amount of sweating of the care recipient 81. Examples of the biometric device 44 include a device that has a Bluetooth, Wi-Fi, or USB communication function and a function to measure biometric information, for example, a general wearable terminal such as Apple Watch provided by Apple Inc., a wearable terminal with a health function, or a wearable terminal for medical use.

The functions illustrated in FIGS. 3 and 5 enable inferring thoughts of the care recipient 81. The functions are described below.

### (Preparation for Data Set)

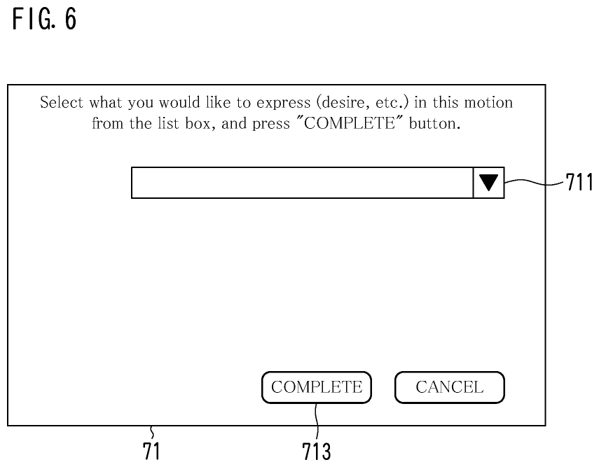

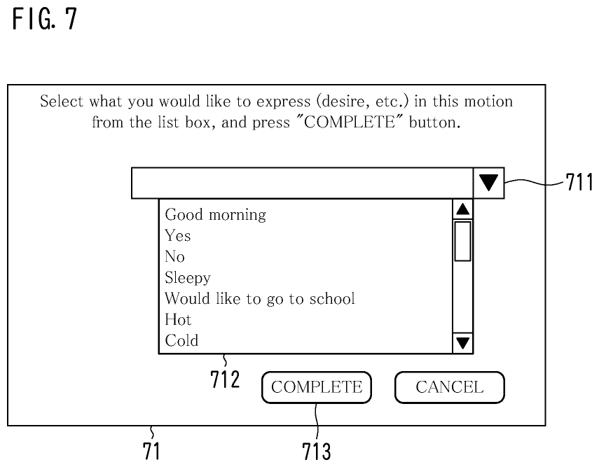

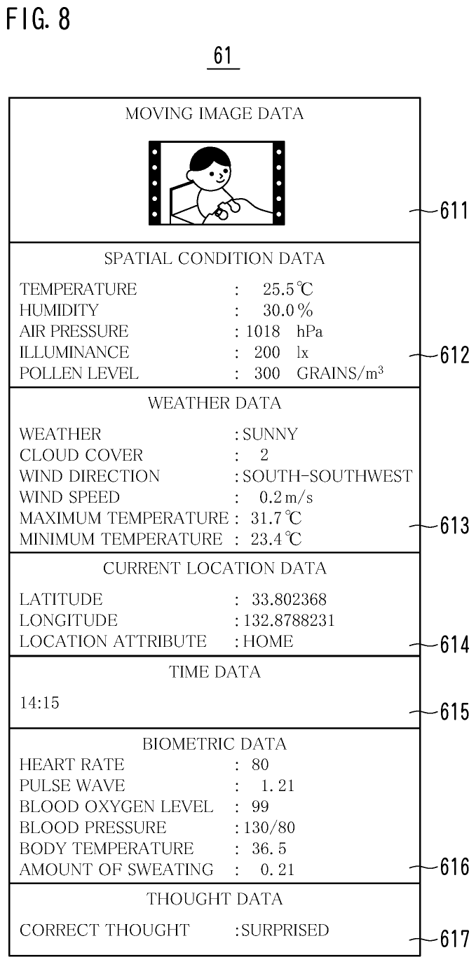

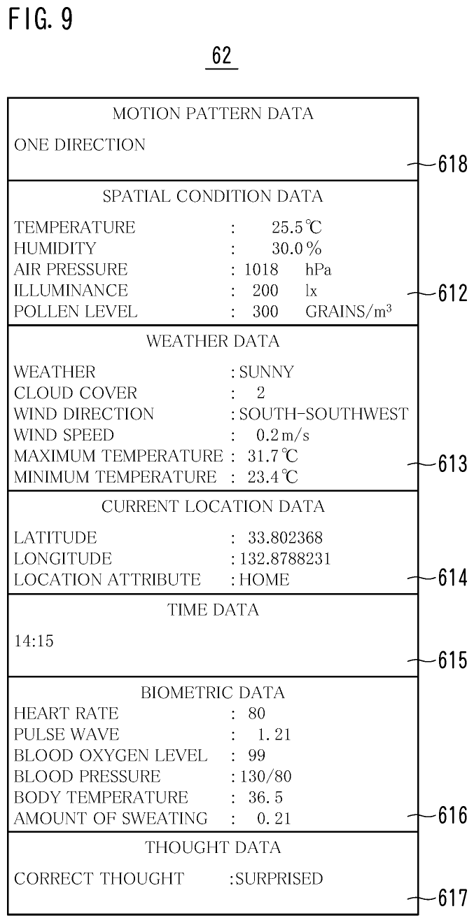

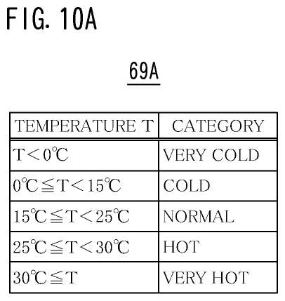

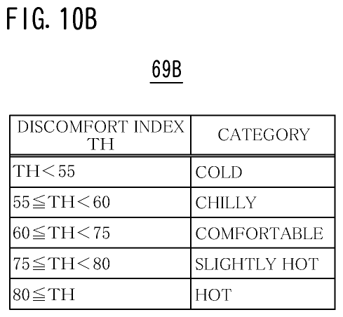

FIG. 6 is a diagram illustrating an example of a thought input screen 71. FIG. 7 is a diagram illustrating an example of an option list 712. FIG. 8 is a diagram illustrating an example of raw data 61. FIG. 9 is a diagram illustrating an example of a data set 62. FIGS. 10A and 10B are diagrams illustrating an example of category tables 69A and 69B.

An operator of the care support server 2 needs to collect a lot of data regarding the care recipient 81 in order to cause the care support server 2 to implement machine learning and generate an inference model. Accordingly, the data is collected with the cooperation of the caregiver 82, with the care recipient 81 regarded as a target for data sampling. Incidentally, each care recipient 81 (81a, 81b, . . . ) is given a unique user code in advance.

One inference model can be shared with a plurality of care recipients 81, but in order to generate a highly accurate inference model with less data, it is desirable to generate an inference model dedicated for each of the care recipients 81. The description goes on to a method for collecting data, taking an example of collecting data for generating an inference model dedicated for the care recipient 81a for a case where a caregiver 82a cares for the care recipient 81a.

The caregiver 82a launches the client program 3P on his/her caregiver terminal 3 and sets the mode to a data collection mode in advance. The data providing module 301 (see FIG. 5) is then activated. The data providing module 301 includes a moving image data extraction module 30A, an environmental data acquisition module 30B, a biometric data acquisition module 30C, a thought data generation module 30D, and a raw data transmission module 30E.

Meanwhile, in general, in a case where a thought such as a desire or a feeling arises, a human sometimes expresses the thought in an action or facial expression. Such a phenomenon is sometimes called a “desire expressive reaction”. The following description takes an example in which the desire expressive reaction appears as a hand gesture.

When the care recipient 81a would like to express his/her thought such as a desire, the care recipient 81a moves his/her hand according to the thought. Then, the video camera 41 installed near the care recipient 81a captures the gesture of the care recipient 81a and also collects sounds around the care recipient 81a. In a case where the care recipient 81a speaks with the gesture, the collected sounds include the voice of the care recipient 81a. The video camera 41 then outputs moving image audio data 601 including a moving image (video) of the captured gesture and the collected sounds to the caregiver terminal 3 of the caregiver 82a. The format of the moving image audio data 601 is, for example, MP4 or MOV.

In a case where the caregiver terminal 3 is put in the data collection mode, in response to the moving image audio data 601 input from the video camera 41, the moving image data extraction module 30A, the environmental data acquisition module 30B, the biometric data acquisition module 30C, the thought data generation module 30D, and the raw data transmission module 30E perform processing as follows.

The moving image data extraction module 30A extracts, from the moving image audio data 601, data corresponding to the moving image as moving image data 611. In other words, data corresponding to the sounds is cut.

The environmental data acquisition module 30B performs processing for acquiring data on the environment around the care recipient 81a as described below. The environmental data acquisition module 30B instructs the indoor measuring instrument 42 near the care recipient 81a to measure the current temperature and so on.

In response to the instructions, the indoor measuring instrument 42 measures the temperature, humidity, air pressure, illuminance, amount of UV radiation, pollen level, and so on, and transmits spatial condition data 612 indicating the measurement result to the caregiver terminal 3.

Further, the environmental data acquisition module 30B accesses an application program interface (API) of a website offering weather information, e.g., OpenWeatherMap API, via the Internet, and acquires weather data 613 indicating information regarding the current weather, cloud cover, wind direction, and wind speed in an area where the care recipient 81a is currently in, and information regarding the maximum temperature, minimum temperature, sunshine duration, and the like in the area of the day. The maximum temperature, the minimum temperature, and the sunshine duration may be actually observed or predicted. The sunshine duration may be calculated based on the sunrise time and the sunset time by the environmental data acquisition module 30B.

The environmental data acquisition module 30B also acquires current location data 614 indicating the current location of the care recipient 81a. Specifically, the latitude and longitude of the caregiver terminal 3 itself is indicated in the current location data 614 as the latitude and longitude of the current location of the care recipient 81a. The latitude and longitude can be acquired with a global positioning system (GPS) function of the caregiver terminal 3 itself.

The current location data 614 also indicates a location attribute of the care recipient 81a. The “location attribute” of the care recipient 81a is an attribute of a facility where the care recipient 81a is currently present. Examples of the attribute include “home”, “classroom”, “dining room”, “bathroom”, “workplace”, “convenience store”, “hospital” and “park”. The location attribute is preferably acquired in the following manner.

For example, for each facility, a table in which latitude and longitude of the facility are correlated with an attribute of the facility is prepared in the client program 3P. The environmental data acquisition module 30B checks the latitude and longitude acquired using the GPS function with the table, and acquires an attribute matching the latitude and longitude as the location attribute of the care recipient 81a.

Alternatively, for each facility, an oscillator for emitting a radio wave indicating a unique identifier is installed in advance and a table in which an identifier of the facility is correlated with an attribute of the facility is prepared in the client program 3P. The environmental data acquisition module 30B checks the identifier indicated in the radio wave received by the short-range wireless board 36 with the table, and acquires an attribute matching the identifier as the location attribute of the care recipient 81a. Examples of such an oscillator include an iBeacon oscillator. “iBeacon” is a registered trademark.

The environmental data acquisition module 30B also acquires time data 615 indicating the current time from a simple network time protocol (SNTP) server. The environmental data acquisition module 30B may acquire the time data 615 from an operating system (OS) or a clock of the caregiver terminal 3 itself. It can be said that the time is a time at which a desire expressive reaction has been observed.

The biometric data acquisition module 30C performs processing for acquiring the current biometric information of the care recipient 81a as follows. The biometric data acquisition module 30C instructs the biometric device 44 of the care recipient 81a to measure the current biometric information. In response to the instructions, the biometric device 44 of the care recipient 81a measures biometric information such as heart rate, pulse wave, blood oxygen level, blood pressure, body temperature, myoelectric potential, amount of sweating, and electrocardiographic potential, and then transmits biometric data 616 indicating the measurement result to the caregiver terminal 3.

The thought data generation module 30D generates thought data 617 indicating, as a correct thought, a thought that the care recipient 81a would like to express, for example, in the following manner.

The thought data generation module 30D displays the thought input screen 71 as illustrated in FIG. 6 on the touch panel display 34. The thought input screen 71 has a list box 711. In response to the caregiver 82a touching the list box 711, the thought data generation module 30D displays an option list 712 directly below the list box 711 as illustrated in FIG. 7. The option list 712 shows character strings representing various thoughts that are preselected by a researcher, a developer, the caregiver 82a, or the like. Hereinafter, such preselected thoughts are referred to as “thought options”. The entirety of the option list 712 cannot be displayed at the same time due to size limitations on the touch panel display 34; however, the caregiver 82a can scroll through the option list 712 to see the option list 712 part by part.

The caregiver 82a determines, based on the motion of the care recipient 81a and the current context (environment such as a time, location, and weather), which thought option matches or is closest to a thought that the care recipient 81a would like to express. The caregiver 82a makes an annotation by scrolling through the option list 712 to touch and select, from the option list 712, a thought option that is determined to match or be closest to the thought that the care recipient 81a would like to express, and touches a complete button 713.

In response to the operation, the thought data generation module 30D generates thought data 617 indicating the selected thought option as a correct thought.

Note that, in a case where the caregiver 82a does not understand or is uncertain about the thought that the care recipient 81a would like to express, the caregiver 82a may make the selection after checking with the care recipient 81a. Even in a case where the caregiver 82a understands the thought that the care recipient 81a would like to express, the caregiver 82a may make the selection after checking with the care recipient 81a just in case. If there are no suitable thought options, then the caregiver 82a may add a thought that the care recipient 81a would like to express as a new thought option, and the thought data generation module 30D may generate thought data 617 indicating the new thought option as a correct thought.

Alternatively, in a case where the care recipient 81a makes a motion, the caregiver 82a may utter a thought option that the caregiver 82a determines to match or be closest to the thought that the care recipient 81a would like to express. In response to the utterance, the voice of the caregiver 82a is recorded in the moving image audio data 601. The thought data generation module 30D then extracts the voice of the caregiver 82a from the moving image audio data 601, and determines the uttered thought option with the speech recognition function. The thought data generation module 30D then generates thought data 617 indicating the determined thought option as a correct thought. Incidentally, in a case where the uttered content (thought) does not correspond to any of the existing thought options, the uttered thought may be added as a new thought option and thought data 617 indicating the added thought option as a correct thought may be generated. Alternatively, AI may classify the uttered thought as any of the existing thought options, and generate, as the thought data 617, data indicating the thought option into which the uttered thought has been classified.

The raw data transmission module 30E correlates the moving image data 611, the spatial condition data 612, the weather data 613, the current location data 614, the time data 615, the biometric data 616, and the thought data 617 with a user code of the care recipient 81a, and transmits, to the care support server 2, the resultant as one set of raw data 61 as illustrated in FIG. 8.

In the care support server 2, the learning module 201 includes a motion pattern determination module 20A, a data set generation module 20B, a data set storage module 20C, a machine learning processing module 20D, and an accuracy test module 20E as illustrated in FIG. 3, and performs processing for generating an inference model as follows.

In a case where the raw data 61 is transmitted from the caregiver terminal 3, the motion pattern determination module 20A determines which of predefined patterns the motion of the care recipient 81a seen in a moving image of the moving image data 611 of the raw data 61 corresponds to, for example, in the following manner.

The motion pattern determination module 20A extracts the position of a hand from each frame of the moving image in the moving image audio data 601 to identify a change in position of the hand, namely, a trajectory of the hand. The motion pattern determination module 20A then determines that, from among the plurality of patterns, a pattern most similar to the identified trajectory corresponds to a pattern of the motion of the care recipient 81a this time.

The trajectory can be identified using a known technique. For example, the trajectory may be identified using Kinovea that is motion analysis software.

Which pattern the identified trajectory corresponds to can also be determined by known techniques. For example, pattern matching may be used for the determination. Alternatively, AI may be used for the determination. Specifically, many trajectories are prepared in advance, and data sets are prepared by an operator labeling which pattern each trajectory corresponds to. Supervised learning is employed to generate a classifier for classifying trajectories. The motion pattern determination module 20A then uses the classifier to determine which pattern the trajectory identified from the moving image data 611 corresponds to.

The predefined patterns are, for example, as follows. “One direction” corresponds to a pattern of moving a hand linearly from a position to another position only once. “Circle” corresponds to a pattern of moving a hand in a circular motion. “Reciprocation” corresponds to a pattern of reciprocating a hand linearly between a position and another position only once. “Weak” corresponds to a pattern of moving a hand in a trembling manner. “Repetition” corresponds to a pattern of making an identical motion twice or more in succession. However, in order to distinguish “repetition” from “weak”, a condition applicable to “repetition” is so set that a hand moves by a certain distance or more (20 cm or more, for example) in one motion. “Random” corresponds to a pattern of moving a hand randomly, which does not correspond to any of the five patterns described above.

The data set generation module 20B generates motion pattern data 618 indicating the determination result by the motion pattern determination module 20A, i.e., the pattern determined, replaces the moving image data 611 of the raw data 61 with the generated motion pattern data 618, and thereby generates the data set 62 as illustrated in FIG. 9. The data set generation module 20B then correlates the generated data set 62 with the user code of the care recipient 81a, and stores the resultant into the data set storage module 20C.

As described above, the motion of the care recipient 81a is triggered to perform the foregoing processing and operation to generate one set of the data set 62, and the data set 62 is stored into the data set storage module 20C. In the preparation phase, every time the care recipient 81a makes a motion, one set of data set 62 is generated by the foregoing processing and operation and is stored into the data set storage module 20C.

Incidentally, as with the case where the motion of the care recipient 81 is classified as any of the plurality of patterns, another piece of information may be classified as any of a plurality of classes and indicated in the data set 62.

For example, it is possible that the data set generation module 20B classifies the temperature indicated in the spatial condition data 612 as any of five categories in accordance with the category table 69A of FIG. 10A. The temperature may be replaced with the classified category. Alternatively, the classified category may be added to the spatial condition data 612.

Alternatively, it is possible that the data set generation module 20B calculates a discomfort index based on the temperature and the humidity indicated in the spatial condition data 612 and classifies the discomfort index as any of five categories in accordance with the category table 69B of FIG. 10B. The classified category may be added to the spatial condition data 612.

Alternatively, it is possible that the data set generation module 20B classifies the time indicated in the time data 615, according to the lifestyle of the care recipient 81a, as any of time frames such as “sleep time frame”, “wake-up time frame”, “breakfast time frame”, “class time frame”, “lunch time frame”, “break time frame”, “free time frame”, “dinner time frame”, and “bedtime frame” based on a timetable prepared in advance. The time may be replaced with the classified time frame. Alternatively, the classified time frame may be added to the time data 615. For further accurate classification, a timetable may be prepared for each day of the week and the time may be classified based on the timetable according to the day of the week at which the time data 615 is acquired.

Yet alternatively, it is possible that the data set generation module 20B calculates a temperature difference between the maximum temperature and the minimum temperature indicated in the weather data 613 and adds the temperature difference to the weather data 613.

Further, in a case where the care recipient 81 (81b, . . . ) other than the care recipient 81a makes a motion, the foregoing processing and operation are also performed to generate each data set 62 for the care recipient 81b, . . . , and that each data set 62 is correlated with the corresponding user code and the resultant is stored into the data set storage module 20C.

### (Generation of Inference Model)

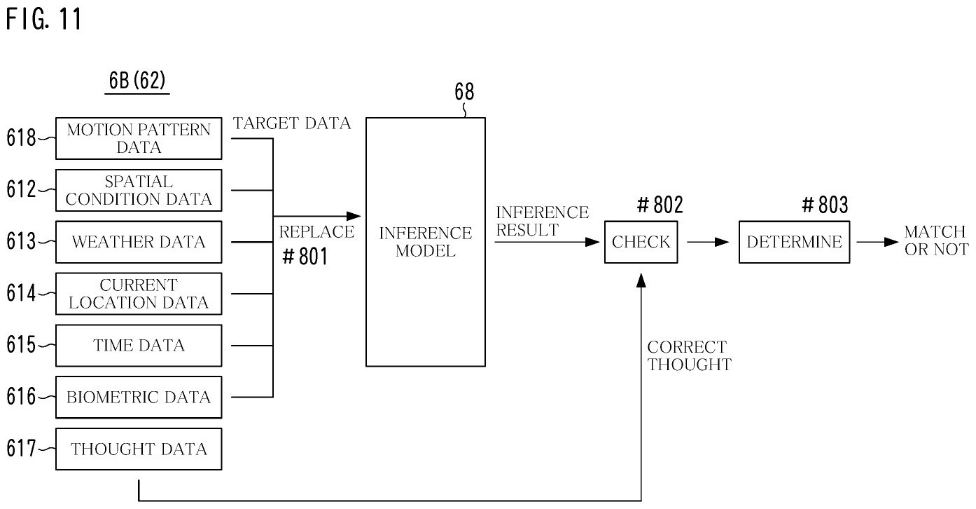

FIG. 11 is a diagram illustrating an example of the flow of processing for testing an inference model 68.

In a case where a certain number of data sets 62 (1,000 sets, for example) or more for a certain care recipient 81 is stored into the data set storage module 20C, the machine learning processing module 20D implements machine learning based on the data sets 62 to generate an inference model for the care recipient 81. The following describes an example in which an inference model for the care recipient 81a is generated.

The machine learning processing module 20D reads data sets 62 correlated with the user code of the care recipient 81a out of the data set storage module 20C. A predetermined ratio (70%, for example) of the data sets 62 is used as training data, and the remaining data sets 62 (30%, for example) are used as test data. Hereinafter, the data set 62 used as the training data is referred to as “training data 6A” and the data set 62 used as the test data is referred to as “test data 6B”.

The machine learning processing module 20D uses, as an objective variable (correct data, label), the thought data 617 of data constituting each set of the training data 6A and uses, as an explanatory variable (input data), the residual data, namely, the spatial condition data 612, the weather data 613, the current location data 614, the time data 615, the biometric data 616, and the motion pattern data 618 to generate the inference model 68 based on a known machine learning algorithm. For example, the machine learning processing module 20D uses a machine learning algorithm such as support vector machine (SVM), random forest, or deep learning to generate the inference model 68. Alternatively, both SVM and BORUTA may be used to generate the inference model 68.

In response to the inference model 68 generated, the accuracy test module 20E uses the test data 6B to test the accuracy of the inference model 68. To be specific, as illustrated in FIG. 11, the spatial condition data 612, the weather data 613, the current location data 614, the time data 615, the biometric data 616, and the motion pattern data 618 of certain test data 6B are input, as target data, to the inference model 68, and thereby the thought that a care recipient would like to express is inferred (#801, #802). To be more specific, any one of the foregoing various thought options is output as an inference result. In a case where the inference model 68 is generated by deep learning, one output node is provided for each thought option and, it is inferred that a thought option of an output node outputting the largest value is the thought that the care recipient would like to express. The same applies to an inference processing module 20H described later.

The accuracy test module 20E determines whether the inferred thought (thought option) matches the correct thought indicated in the thought data 617 (#803). Other test data 6B also undergoes the processing of steps #801 to #803.

If the number of times that the correct thought matches the inferred thought in step #803 with respect to the number of times the processing of steps #801 to #803 has been performed is a predetermined ratio (for example, 90%) or more, then the accuracy test module 20E certifies that the inference model 68 is acceptable, and the inference model 68 is correlated with the user code of the care recipient 81a and the resultant is stored into the inference model storage module 202. Thereby, preparation for inferring the thought of the care recipient 81a is completed.

On the other hand, if the number of times that the correct thought matches the inferred thought in step #803 with respect to the number of times the processing of steps #801 to #803 has been performed is smaller than the predetermined ratio, then the accuracy test module 20E discards the inference model 68 and the collection of the data set 62 is continued. Alternatively, an administrator may adjust the target data by changing the value of the weight parameter of any of the items (for example, weather of the weather data 613, or location attribute of the current location data 614) constituting each of the target data (spatial condition data 612, weather data 613, current location data 614, time data 615, biometric data 616, and motion pattern data 618), or, alternatively, by deleting any of the items (for example, cloud cover of the weather data 613, or latitude and longitude of the current location data 614) and may conduct a test again.

An inference model 68 for the care recipient 81 (81b, . . . ) other than the care recipient 81a is generated in the similar method, and the inference model 68 is correlated with the corresponding user code and the resultant is stored in the inference model storage module 202.

### (Thought Inference)

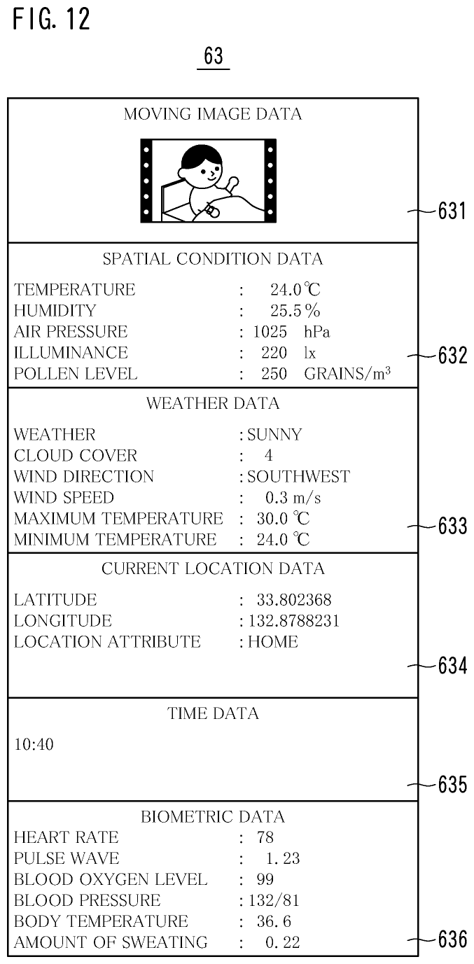

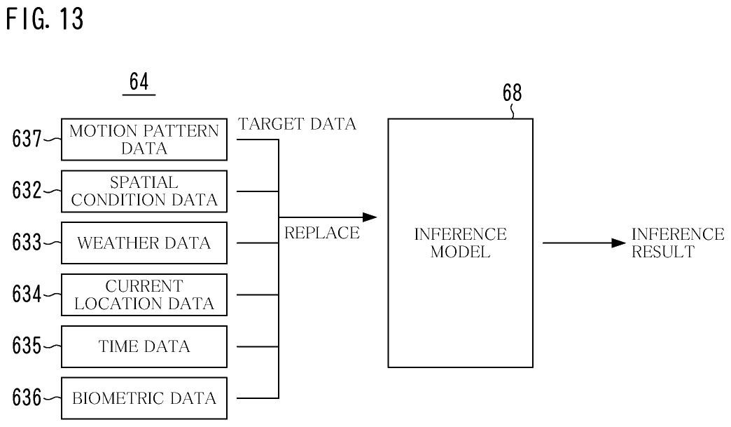

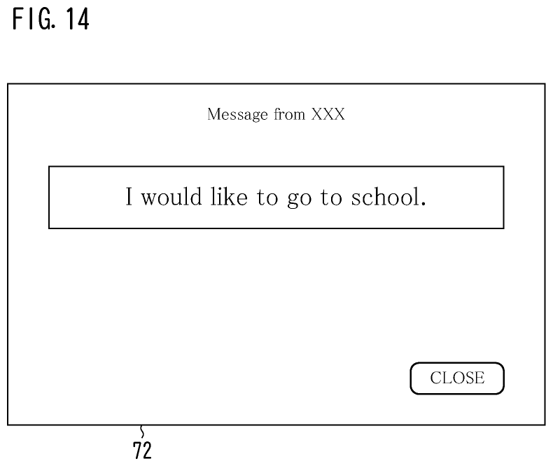

FIG. 12 is a diagram illustrating an example of raw data 63. FIG. 13 is a diagram illustrating an example of the flow of inference processing. FIG. 14 is a diagram illustrating an example of an inference result screen 72.

In a case where an inference model 68 for a certain care recipient 81 is stored into the data set storage module 20C, the inference module 203 of the care support server 2 and the client module 302 of the caregiver terminal 3 are ready to infer a thought of the care recipient 81. The description goes on to an inference method by taking an example in which the video camera 41 captures an image of the care recipient 81a to infer a thought of the care recipient 81a in a case where the caregiver 82a cares for the care recipient 81a.

The caregiver 82a launches the client program 3P on his/her caregiver terminal 3 and sets the mode to an inference mode in advance. The client module 302 is then activated. As illustrated in FIG. 5, the client module 302 includes a moving image data extraction module 30F, an environmental data acquisition module 30G, a biometric data acquisition module 30H, a raw data transmission module 30J, and an inference result output module 30K.

When the care recipient 81a makes a gesture in order to express his/her thought to the caregiver 82a, the video camera 41 installed near the care recipient 81a captures the gesture of the care recipient 81a and also collects sounds around the care recipient 81a. Thereby, the moving image audio data 601 is generated. Then, the moving image audio data 601 is output from the video camera 41 to be input to the caregiver terminal 3 of the caregiver 82a.

In a case where the caregiver terminal 3 is put in the data collection mode, in response to the moving image audio data 601 input from the video camera 41, the moving image data extraction module 30F, the environmental data acquisition module 30G, the biometric data acquisition module 30H, and the raw data transmission module 30J perform processing as follows.

The moving image data extraction module 30F extracts, from the moving image audio data 601 input this time, data corresponding to the moving image as moving image data 631. In other words, as with the moving image data extraction module 30A of the data providing module 301, the moving image data extraction module 30F cuts data corresponding to the sounds.

The environmental data acquisition module 30G acquires the following data in a manner similar to that of the processing of the environmental data acquisition module 30B. The environmental data acquisition module 30G instructs the indoor measuring instrument 42 near the care recipient 81a to measure the current temperature and so on, so that spatial condition data 632 indicating the current temperature, humidity, air pressure, illuminance, amount of UV radiation, and pollen level is acquired. The environmental data acquisition module 30G acquires, from a website, weather data 633 indicating information regarding the current weather, cloud cover, wind direction, and wind speed in an area where the care recipient 81a is currently in, and information regarding the maximum temperature, minimum temperature, and the like in the area of the day. The environmental data acquisition module 30G acquires current location data 634 indicating the latitude and longitude of the current location of the care recipient 81a and the location attribute by using the GPS function or iBeacon. The environmental data acquisition module 30G acquires time data 635 indicating the current time from the SNTP server or the like.

The biometric data acquisition module 30H acquires, from the biometric device 44 of the care recipient 81a, biometric data 636 indicating biometric information such as the current heart rate, pulse wave, blood oxygen level, blood pressure, electrocardiographic potential, body temperature, myoelectric potential, and amount of sweating in a manner similar to that of the processing of the biometric data acquisition module 30C.

The raw data transmission module 30J correlates the moving image data 631, the spatial condition data 632, the weather data 633, the current location data 634, the time data 635, and the biometric data 636 with the user code of the care recipient 81a to transmit the resultant as one set of raw data 63 illustrated in FIG. 12 to the care support server 2.

In the care support server 2, the inference module 203 incudes a motion pattern determination module 20F, a target data generation module 20G, an inference processing module 20H, and an inference result replying module 20J as illustrated in FIG. 3, and infers a thought of the care recipient 81a in the following manner.

In a case where the raw data 63 is transmitted from the caregiver terminal 3, the motion pattern determination module 20F determines which of the predefined patterns the current motion of the care recipient 81a seen in a moving image of the moving image data 631 of the raw data 63 corresponds to. The determination method is similar to the determination method of the motion pattern determination module 20A.

The target data generation module 20G generates motion pattern data 637 indicating the determination result by the motion pattern determination module 20F, i.e., the pattern determined, replaces the moving image data 631 of the raw data 63 with the generated motion pattern data 637, and thereby generates target data 64.

The inference processing module 20H infers, as illustrated in FIG. 13, the thought of the care recipient 81a by inputting the generated target data 64 to the inference model 68 correlated with the user code of the care recipient 81a and stored in the inference model storage module 202 to acquire an output value from the inference model 68. As described above, in a case where the inference model 68 is generated by deep learning, the inference processing module 20H infers that a thought option of an output node outputting the largest value is the thought of the care recipient 81a.

The inference result replying module 20J transmits inference result data 65 indicating the thought of the care recipient 81a inferred by the inference processing module 20H to the caregiver terminal 3 of the caregiver 82a.

In the caregiver terminal 3, in response to the inference result data 65 received, the inference result output module 30K (see FIG. 5) displays a character string representing the thought indicated in the inference result data 65 in the form of the inference result screen 72 illustrated in FIG. 14 on the touch panel display 34, and a voice representing the thought is reproduced by the speech processing module 39.

Further, it is possible to cause the smart speaker 43 to hear the voice reproduced by the speech processing module 39 to deal with a desire of the care recipient 81a. For example, in a case where the voice “hot” is input, the smart speaker 43 turns on an air conditioner of a room where the care recipient 81a is present, or lowers the set temperature of the air conditioner. Further, in a case where the voice “Where is the teacher?” is input, the smart speaker 43 generates an email with content of “Where is the teacher?” and sends the email to a school teacher of the care recipient 81a.

A thought of the care recipient 81 (81b, . . . ) other than the care recipient 81a is also inferred based on the corresponding raw data 63 and inference model 68 in the similar method, and the inference result is sent to the caregiver terminal 3 of the caregiver 82 who cares for the corresponding care recipient 81 (81b, . . . ).

### (Flow of Entire Processing of Care Support Server 2)

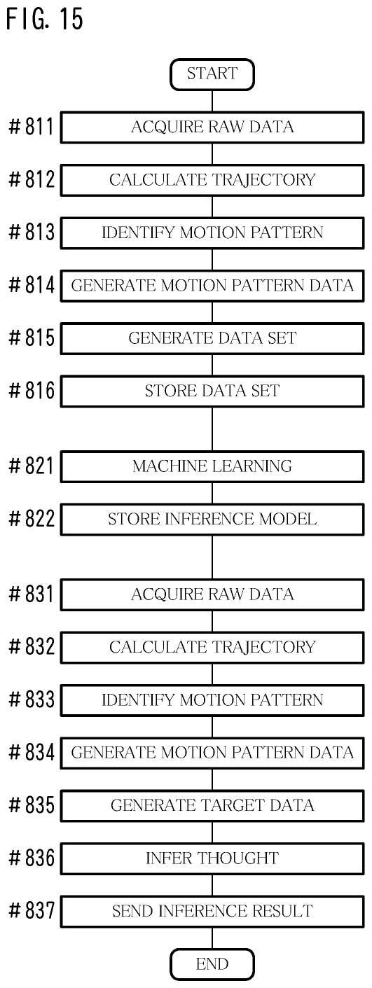

FIG. 15 is a flowchart depicting an example of the flow of the entire processing by the inference service program 2P.

The description goes on to the flow of the entire processing in the care support server 2 with reference to the flowchart.

The care support server 2 executes the processing based on the inference service program 2P in the steps depicted in the flowchart of FIG. 15.

In the data set preparation phase, the care support server 2 receives the raw data 61 (see FIG. 8) from the caregiver terminal 3 (#811), calculates the trajectory of a hand of the care recipient 81 based on the moving image data 611 of the raw data 61 (#812), and identifies a motion pattern represented by the trajectory (#813). Note that the raw data 61 is correlated with a user code.

The care support server 2 then generates the motion pattern data 618 indicating the identified pattern (#814), replaces the moving image data 611 with the motion pattern data 618 to generate the data set 62 (see FIG. 9) (#815), and correlates the data set 62 with the user code to store the resultant data set 62 (#816).

Every time the raw data 61 is received, the care support server 2 executes the processing of steps #812 to #816.

In the machine learning phase, when the number of data sets 62 correlated with an identical user code reaches a predetermined number of sets, the care support server 2 generates an inference model 68 by implementing machine learning based on these data sets 62 (#821). In a case where a test confirms that the inference model 68 has a certain degree of accuracy, the inference model 68 is correlated with the user code and stored (#822).

In the inference phase, when the raw data 63 (see FIG. 12) is received from the caregiver terminal 3 (#831), the care support server 2 calculates the trajectory of the hand of the care recipient 81 based on the moving image data 631 of the raw data 63 (#832) to identify a motion pattern represented by the trajectory (#833). Note that the user code is correlated with the raw data 63.

The care support server 2 generates the motion pattern data 637 indicating the identified pattern (#834), replaces the moving image data 631 with the motion pattern data 637 to generate the target data 64 (#835).

The care support server 2 then inputs the target data 64 to an inference model 68 correlated with the user code to infer the thought of the care recipient 81 (#836), and transmits the inference result data 65 indicating the inference result to the caregiver terminal 3 (#837). In response to the operation, the inference result is output in the form of a character string or a voice in the caregiver terminal 3.

Every time the raw data 63 is received, the care support server 2 executes the processing of steps #832 to #837.

According to the present embodiment, in a case where the care recipient 81 has a thought such as a desire or something that the care recipient 81 would like to express, the thought can be inferred and output even when the caregiver 82 is not present. This makes it possible for the care recipient 81 to use an ICT device while the burden on the caregiver 82 is reduced as compared with the conventional cases. Even in a case where the care recipient 81 is a person with severe motor and intellectual disabilities, a dementia patient, or the like, the ICT device can be used more easily than conventionally possible.

### (Modification)

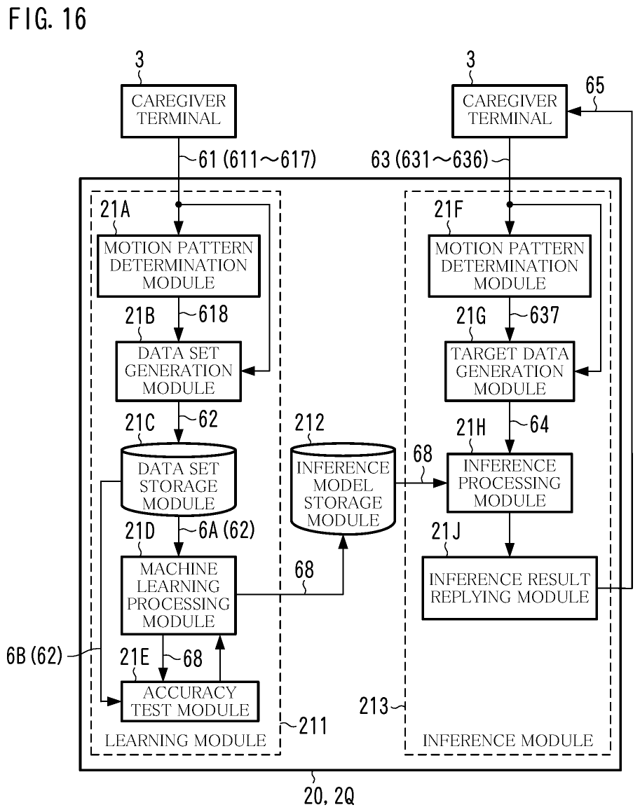

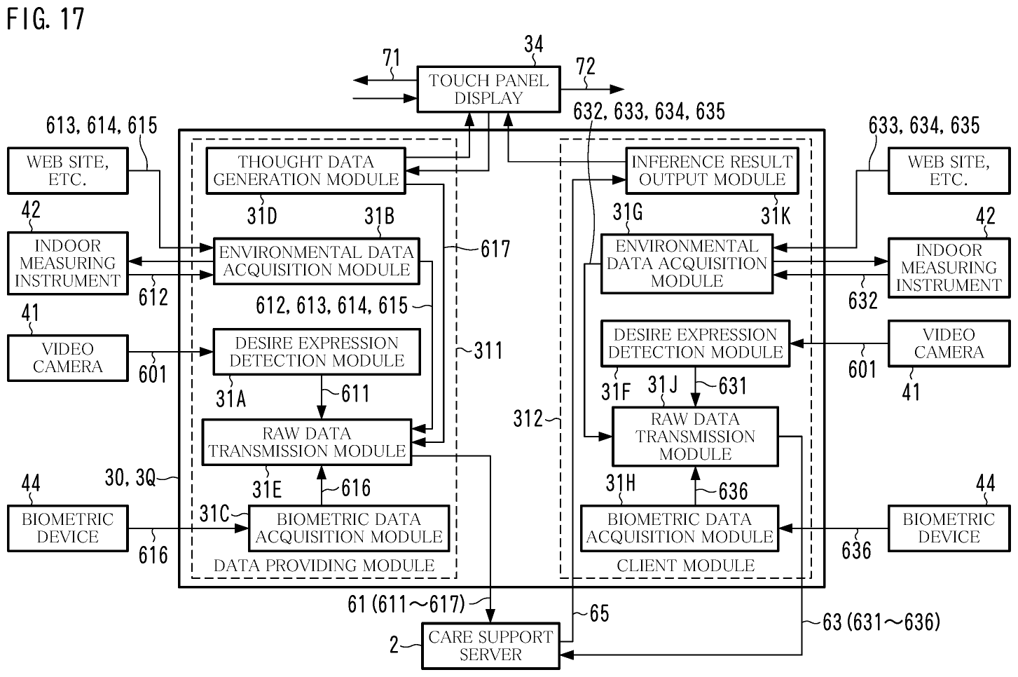

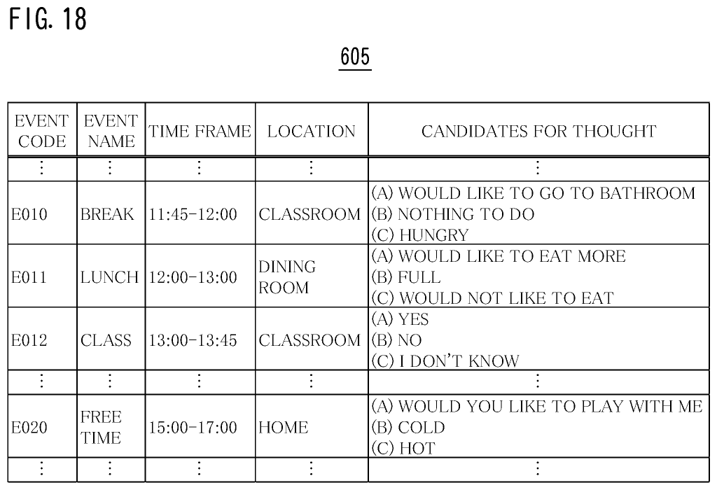

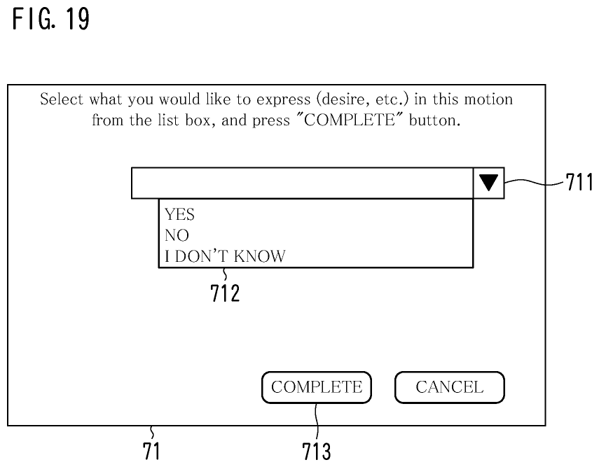

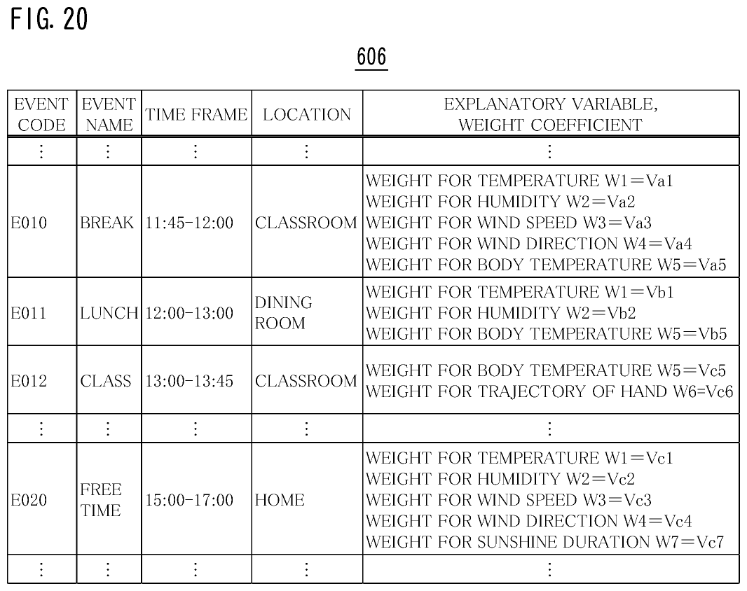

FIG. 16 is a diagram illustrating a modification to the functional configuration of the care support server 2. FIG. 17 is a diagram illustrating a modification to the functional configuration of the caregiver terminal 3. FIG. 18 is a diagram illustrating an example of an event table 605. FIG. 19 is a diagram illustrating a modification to the option list 712. FIG. 20 is a diagram illustrating an example of an explanatory variable table 606.

In the present embodiment, the care support server 2 implements machine learning by using the data set 62, specifically, the data on the items illustrated in FIG. 9, as training data. The care support server 2 may implement machine learning by adding data on other items to the data set 62. The same applies to the inference.

For example, the care support server 2 may implement machine learning and inference by adding data indicating 3-axis geomagnetism and 3-axis acceleration of the current location of the care recipient 81 to the data set 62 and the target data 64, respectively.

In the present embodiment, the data set 62 and the target data 64 include, as data indicating a desire expressive reaction of the care recipient 81, the motion pattern data 618 and the motion pattern data 637, respectively. Specifically, data indicating a pattern of a hand gesture of the care recipient 81 is included in the data set 62 and the target data 64, and the care support server 2 implements machine learning and inference based on the pattern. Instead of this, however, the care support server 2 may implement machine learning and inference based on another desire expressive reaction. For example, the care support server 2 may implement machine learning and inference based on a pattern of eye movements, a pattern of eyelid movements (open/close), a pattern of change in voice pitch, presence/absence of uttering, a pattern of facial expressions, or the like of the care recipient 81. Alternatively, the care support server 2 may implement machine learning and inference based on a plurality of desire expressive reactions (a pattern of hand gestures and a pattern of facial expressions) arbitrarily selected from among the desire expressive reactions described above. The same applies to a modification described later with reference to FIGS. 16-20.

Incidentally, the eye movement, the eyelid movement, and the facial expression are preferably extracted from a moving image captured by the video camera 41 or the video camera 37 of the caregiver terminal 3. Alternatively, the care recipient 81 may wear a glass wearable terminal to capture the eye movement and the eyelid movement with the wearable terminal. The voice pitch and the presence/absence of uttering are preferably identified based on audio data included in the moving image audio data 601.

In the present embodiment, the care support server 2 identifies the trajectory of a hand of the care recipient 81 based on a moving image. Instead of this, however, the care support server 2 may identify the trajectory based on data on a posture, angular velocity, or angular acceleration acquired by a gyro sensor of the biometric device 44 attached to a wrist of the care recipient 81. The same applies to the modification described later with reference to FIGS. 16-20.

The care support server 2 may include, in the data set 62 (see FIG. 9), only data on some items indicated in the raw data 61 (see FIG. 8) instead of including data on all the items indicated therein. For example, the data set 62 may be generated in a manner to include only the following: the current location data 614, the time data 615, and the thought data 617 of the raw data 61; data on temperature and humidity of the spatial condition data 612; data on wind direction and wind speed of the weather data 613; and data on body temperature of the biometric data 616.

In the present embodiment, the care support server 2 generates one inference model 68 for one care recipient 81. Instead of this, however, the care support server 2 may generate one inference model 68 for one care recipient 81 for each time frame and for each location. The following description takes an example in which an inference model 68 for the care recipient 81a is generated.

The care support server 2 has installed, thereon, an inference service program 2Q instead of the inference service program 2P. The inference service program 2Q enables implementing the functions of a learning module 211, an inference model storage module 212, and an inference module 213 illustrated in FIG. 16.

The learning module 211 includes a motion pattern determination module 21A, a data set generation module 21B, a data set storage module 21C, a machine learning processing module 21D, and an accuracy test module 21E, and executes processing for generating an inference model.

The inference module 213 includes a motion pattern determination module 21F, a target data generation module 21G, an inference processing module 21H, and an inference result replying module 21J, and executes processing for inferring a thought.

The caregiver terminal 3 has installed, thereon, a client program 3Q instead of the client program 3P. The client program 3Q enables implementing the functions of a data providing module 311, a client module 312, and so on illustrated in FIG. 17.

The data providing module 311 includes a desire expression detection module 31A, an environmental data acquisition module 31B, a biometric data acquisition module 31C, a thought data generation module 31D, and a raw data transmission module 31E, and provides the care support server 2 with data for learning.

The client module 312 includes a desire expression detection module 31F, an environmental data acquisition module 31G, a biometric data acquisition module 31H, a raw data transmission module 31J, and an inference result output module 31K, and causes the care support server 2 to implement inference.

When the care recipient 81a moves his/her hand in order to express his/her thought such as a desire, the video camera 41 near the care recipient 81a captures the motion of the care recipient 81a and collect sounds, and the moving image audio data 601 is output to the caregiver terminal 3 of the caregiver 82a.

In the caregiver terminal 3 placed in the data collection mode, in a case where the moving image audio data 601 is input from the video camera 41, the desire expression detection module 31A determines whether a desire expressive reaction is seen based on the moving image audio data 601, for example, in the following manner.

If the hand gesture indicated in the moving image audio data 601 is a predetermined gesture (for example, a motion of reciprocating the hand between two points in succession), then it is determined that a desire expressive reaction is seen. Alternatively, if the voice indicated in the moving image audio data 601 continues for a predetermined time (two seconds, for example) or more, it may be determined that a desire expressive reaction is seen.

In a case where the desire expression detection module 31A determines that a desire expressive reaction is seen, the environmental data acquisition module 31B and the biometric data acquisition module 31C perform the following processing.

The environmental data acquisition module 31B acquires data regarding the environment around the care recipient 81a, namely, the spatial condition data 612, the weather data 613, the current location data 614, and the time data 615. The acquisition method is similar to the method for acquiring these sets of data by the environmental data acquisition module 30B (see FIG. 5).

The biometric data acquisition module 31C acquires data on the current biometric information of the care recipient 81a, namely, the biometric data 616. The acquisition method is similar to the method for acquiring the data by the biometric data acquisition module 30C.

The desire expression detection module 31A extracts the moving image data 611 from the moving image audio data 601, as with the moving image data extraction module 30A.

The thought data generation module 31D has the event table 605 for the care recipient 81a. As illustrated in FIG. 18, the event table 605 indicates, for each event (activity) such as a daily break, lunch, class, free time, dinner, bath, stroll, and bedtime of the care recipient 81a, a time frame and location in which the subject event occurs. The location may be represented by the latitude and longitude, or, by a location attribute (for example, “home”, “classroom”, “dining room”, or “bathroom”). Further, the event table 605 indicates, as thought options, a plurality of thoughts that the care recipient 81a may have during the subject event. The thought options are determined in advance by a researcher or a developer in consultation with the caregiver 82a. Note that the thought data generation module 31D has the event table 605 for each of the other care recipients 81 (81b, . . . ).

The thought data generation module 31D generates thought data 617 indicating, as a correct thought, a thought that the care recipient 81a would like to express based on the event table 605 and so on, for example, in the following manner.

The thought data generation module 31D determines, based on the event table 605, an event that corresponds to the current location indicated in the current location data 614 and the time indicated in the time data 615. For example, in a case where the current location data 614 indicates “classroom” and the time data 615 indicates “11:50”, the thought data generation module 31D determines that the event is “class”.

In response to the event determined, the thought data generation module 31D displays the thought input screen 71 (see FIG. 6) on the touch panel display 34. In response to the caregiver 82a touching the list box 711, the thought data generation module 31D displays the option list 712 directly below the list box 711 as illustrated in FIG. 19. The option list 712 has a plurality of thought options that is correlated with the determined event in the event table 605.

The caregiver 82a determines, based on the motion, the state, and so on of the care recipient 81a, which thought option matches or is closest to a thought that the care recipient 81a would like to express. The caregiver 82a then touches and selects, from the option list 712, a thought option that is determined to match or be closest to the thought that the care recipient 81a would like to express, and touches the complete button 713.

In response to the operation, the thought data generation module 31D generates thought data 617 indicating the selected thought option as a correct thought. Note that if there are no suitable thought options, a new thought option may be added as with the thought data generation module 30D.

The raw data transmission module 31E correlates the moving image data 611, the spatial condition data 612, the weather data 613, the current location data 614, the time data 615, the biometric data 616, and the thought data 617 with an event code of the event this time and the user code of the care recipient 81a, and transmits, to the care support server 2, the resultant as one set of raw data 61.

In the care support server 2, in a case where the raw data 61 is transmitted from the caregiver terminal 3, the motion pattern determination module 21A determines which of the predefined patterns the motion of the care recipient 81a seen in a moving image of the moving image data 611 of the raw data 61 corresponds to. The determination method is similar to the determination method of the motion pattern determination module 20A illustrated in FIG. 3.

The data set generation module 21B generates the data set 62 (see FIG. 9) based on the determination result of the motion pattern determination module 21A as with the data set generation module 20B. The data set generation module 21B then correlates the generated data set 62 with the user code of the care recipient 81a and the event code of the event this time, and stores the resultant into the data set storage module 21C.

The machine learning processing module 21D has the explanatory variable table 606 for the care recipient 81a. As illustrated in FIG. 20, the explanatory variable table 606 indicates, for each event in the daily life of the care recipient 81a, one or a plurality of explanatory variables used for machine learning and each weight coefficient regarding the subject event. The explanatory variables are determined in advance, by the researcher or the developer in consultation with the caregiver 82a, from variables included in each of the spatial condition data 612, the weather data 613, the biometric data 616, and the motion pattern data 618 based on the daily situation of the care recipient 81a. Further, as with the event table 605 (see FIG. 18), a time frame and a location in which each event occurs are indicated. The machine learning processing module 21D also has the explanatory variable table 606 for each of the other care recipients 81 (81b, . . . ).

In a case where a certain number of data sets 62 (100 sets, for example) or more that is correlated with the user code of the care recipient 81a and correlated with an identical event code is stored into the data set storage module 21C, the machine learning processing module 21D reads the data sets 62 out of the data set storage module 21C. A predetermined ratio of the data sets 62 is used as the training data 6A and the remaining data sets 62 are used as the test data 6B.

The machine learning processing module 21D uses the training data 6A to generate an inference model 68 for the event of the care recipient 81a based on a known machine learning algorithm. The point that the thought data 617 of the data constituting each set of the training data 6A is used as the objective variable is similar to machine learning by the machine learning processing module 20D. However, among variables included in each set of the spatial condition data 612, the weather data 613, the current location data 614, the time data 615, the biometric data 616, and the motion pattern data 618, a variable correlated with the event in the explanatory variable table 606 of the care recipient 81a is selected and used as the explanatory variable. In other words, the machine learning processing module 21D implements machine learning with the thought data 617 and the data indicating the selected explanatory variable used as the data set. Further, at this time, a weight coefficient of each variable (explanatory variable) indicated in the explanatory variable table 606 is used.

Incidentally, the explanatory variable used in machine learning may be selected by not the machine learning processing module 21D but the data set generation module 21B. To be specific, in response to the raw data 61 transmitted from the caregiver terminal 3, the data set generation module 21B selects an explanatory variable corresponding to the event code of the raw data 61 based on the explanatory variable table 606 for the care recipient 81a. The data set generation module 21B then combines the thought data 617 with data on the selected explanatory variable to store the resultant as the data set 62 into the data set storage module 21C.

In response to the inference model 68 generated, the accuracy test module 21E uses the test data 6B to test the accuracy of the inference model 68. The test method is basically similar to the test method by the accuracy test module 20E. It should be noted that the explanatory variable indicated in the explanatory variable table 606 is used.

In a case where the accuracy test module 21E certifies that the inference model 68 is acceptable, the inference model 68 is correlated with the user code of the care recipient 81a and the event code of the corresponding event, and the resultant is stored into the inference model storage module 212.

The individual modules of the inference module 213 of the care support server 2 and the individual modules of the client module 312 of the caregiver terminal 3 perform processing for inferring a thought of the care recipient 81. Hereinafter, the processing is described by taking an example of inferring a thought of the care recipient 81a.

In the caregiver terminal 3 placed in the inference mode, in a case where the moving image audio data 601 is input from the video camera 41, the desire expression detection module 31F determines whether a desire expressive reaction is seen based on the moving image audio data 601. The determination method is similar to the determination method by the desire expression detection module 31A. The desire expression detection module 31F also extracts the moving image data 631 as with the moving image data extraction module 30F (see FIG. 5).

If it is determined that a desire expressive reaction is seen, then the environmental data acquisition module 31G and the biometric data acquisition module 31H perform the following processing.

The environmental data acquisition module 31G acquires the spatial condition data 632, the weather data 633, the current location data 634, and the time data 635 in a method similar to the processing method of the environmental data acquisition module 30B.

The biometric data acquisition module 31H acquires the biometric data 636 in a method similar to the processing method of the biometric data acquisition module 30C.

The raw data transmission module 31J correlates the moving image data 631, the spatial condition data 632, the weather data 633, the current location data 634, the time data 635, and the biometric data 636 with the user code of the care recipient 81a, and transmits, to the care support server 2, the resultant as one set of raw data 61.

In the care support server 2, the motion pattern determination module 21F determines which of the patterns a motion of the care recipient 81a corresponds to, in a method similar to the method of the motion pattern determination module 20A.

The target data generation module 21G generates the motion pattern data 637 indicating the determination result of the motion pattern determination module 21F, and replaces the moving image data 631 of the raw data 61 with the generated motion pattern data 637, and thereby generates the target data 64.

As with the machine learning processing module 21D, the inference processing module 21H includes the explanatory variable table 606 (see FIG. 20).

The inference processing module 21H determines, based on the explanatory variable table 606, an event that corresponds to a current location indicated in the current location data 634 and corresponds to a time indicated in the time data 635 of the target data 64. For example, in a case where the current location data 634 indicates “classroom” and the time data 635 indicates “11:55”, the inference processing module 21H determines that the event is “break”.

The inference processing module 21H inputs a value of a thought option corresponding to the determined event in the target data 64 to an inference model 68 corresponding to the user code of the care recipient 81a and the event code of the event, acquires an output value from the inference model 68, and infers the thought of the care recipient 81a.

The inference result replying module 21J transmits, as the inference result data 65, data indicating the thought of the care recipient 81a inferred by the inference processing module 21H to the caregiver terminal 3 of the caregiver 82a.

In the caregiver terminal 3, in response to the inference result data 65 received, the inference result output module 31K displays a character string representing the thought indicated in the inference result data 65 in the form of the inference result screen 72 (see FIG. 14) on the touch panel display 34, and a voice representing the thought is reproduced by the speech processing module 39. Then, it is possible to cause the smart speaker 43 to hear the voice to deal with a desire of the care recipient 81a.

In the caregiver terminal 3, in response to the inference result data 65 received, the inference result output module 31K (see FIG. 17) displays a character string representing the thought indicated in the inference result data 65 on the touch panel display 34, and a voice representing the thought is reproduced by the speech processing module 39.

A thought of the care recipient 81 (81b, . . . ) other than the care recipient 81a is also inferred based on the corresponding target data 64 and inference model 68 in the similar method, and the inference result is sent to the caregiver terminal 3 of the caregiver 82 who cares for the corresponding care recipient 81 (81b, . . . ).

According to the method for implementing machine learning by preselecting, for each event, an explanatory variable to be used as described above, it is possible to generate a fitted inference model 68 for each event, which increases the accuracy of inference. One of the main factors that the inventor was able to devise this method is that variables highly correlated with the desire expressive reaction were identified from among the foregoing various variables by experiments.

The inventor calculated, by experiments, correlations between thoughts of the care recipient 81 and various variables regarding the environment or body of the care recipient 81 (for example, temperature, humidity, air pressure, illuminance, amount of UV radiation, pollen level, weather, cloud cover, wind direction, wind speed, maximum temperature, minimum temperature, sunshine duration, temperature difference, current location, time, time frame, 3-axis geomagnetism, 3-axis acceleration, heart rate, pulse wave, blood oxygen level, blood pressure, body temperature, amount of sweating, electrocardiographic potential, myoelectric potential, motion pattern, eye movement, eyelid movement, change in voice pitch, presence/absence of uttering, pattern of facial expressions, and so on). The experiments have shown that, in particular, thoughts of the care recipient 81 are easy to change in different current locations, i.e., in different places, and further, thoughts of the care recipient 81 are easy to change in different times or time frames. In short, the experiments have shown that the location and the time are highly correlated with the desire expressive reaction.

Further, the inventor identified that a combination of location and time corresponds to an event in the daily life of the care recipient 81. In view of this, the inference model 68 was generated for each combination of location and time, namely, for each event, as described in the modification.

Further, the experiments by the inventor have shown that a variable highly correlated with the desire expressive reaction among the various variables is different depending on location or time. As examples of the explanatory variable used in machine learning, the event table 605 (see FIG. 18) and the explanatory variable table 606 (see FIG. 20) are taken. The examples were selected based on the experiments and researches conducted by the inventor at a certain school for the disabled. The explanatory variable used in machine learning is preferably set for each care recipient 81 according to the context (environment) of the care recipient 81. For example, a blood oxygen level may be included as an explanatory variable for an event where the care recipient 81 may call someone. In particular, that is suitable for a case where the care recipient 81 is on a respirator.

In the examples of FIGS. 3, 5, 16, and 17, the care support server 2 performs the processing for generating the data set 62 from the raw data 61. Instead of this, the caregiver terminal 3 may perform the processing. Similarly, the caregiver terminal 3 may perform the processing for generating the target data 64 from the raw data 63.

In the examples of FIGS. 3 and 16, the care support server 2 implements machine learning and inference. Instead of this, different servers may implement machine learning and inference. For example, a machine learning server may implement machine learning and an inference server may implement inference. In such a case, after generating an inference model 68, the machine learning server sends the inference model 68 to the inference server and install the inference model 68 in the inference server. Alternatively, in a case where the inference model 68 is a trained model generated by deep learning, only parameters of the inference model 68 may be sent to the inference server and the parameters may be applied to the inference model prepared in the inference server. The caregiver terminal 3 sends the raw data 61 to the machine learning server and sends the raw data 63 to the inference server.

In the examples of FIGS. 3 and 16, the data set 62 is stored into the care support server 2. Instead of this, the data set 62 may be stored into another server. For example, the data set 62 may be stored into a cloud server of Japan Gigabit Network (JGN) of National Institute of Information and Communication Technology (NICT). A cloud server having a machine learning engine may implement machine learning.

In the present embodiment, the inference model 68 is generated and used properly for each care recipient 81. Instead of this, a common inference model 68 may be generated and shared with a plurality of care recipients 81.

The configuration, the processing contents, the processing sequence, the configuration of the data set, and so on of the entire or each part of the thought inference system 1, the care support server 2, and the caregiver terminal 3 can be changed appropriately according to the gist of the present invention.

1. A thought inference system comprising:
a data set acquisition module configured to acquire a plurality of data sets, each of the data sets indicating a first condition for a case where a first physical reaction is seen in a person with speech difficulties and a first thought of the person with speech difficulties for a case where the first physical reaction is seen;
an inference model generation module configured to generate, for each of a plurality of combinations of a time frame and a location, an inference model by machine learning in which the first condition indicated in the data set acquired in a time frame and a location of the subject combination is used as an explanatory variable and the first thought indicated in the data set is used as an objective variable;
an inference module configured to infer a second thought for a case where a second reaction is seen in the person with speech difficulties by inputting input data indicating a second condition for a case where the second reaction is seen to the inference model that is generated, among the plurality of combinations, for a combination of a time frame and a location in which the second reaction is seen; and
an output module configured to output the second thought.

2. An inference model generation system comprising:
a data set acquisition module configured to acquire a plurality of data sets, each of the data sets indicating a condition for a case where a physical reaction is seen in a person with speech difficulties and a thought of the person with speech difficulties for a case where the physical reaction is seen; and
an inference model generation module configured to generate, for each of a plurality of combinations of a time frame and a location, an inference model by machine learning in which the condition indicated in the data set acquired in a time frame and a location of the subject combination is used as an explanatory variable and the thought indicated in the data set is used as an objective variable.

3. The inference model generation system according to claim 2, wherein the data set acquisition module acquires, as the data set, data indicating biometric information of the person with speech difficulties or a state around the person with speech difficulties.

4. The inference model generation system according to claim 3, wherein the data set acquisition module acquires, as the data set, data indicating weather around the person with speech difficulties.

5. The inference model generation system according to claim 3, wherein
the data set acquisition module acquires, as the data set, data indicating information on each of a plurality of items, and
the inference model generation module generates the inference model by using a weight coefficient set in each of the plurality of items.

6. The inference model generation system according to claim 5, wherein the condition includes a condition regarding a motion of the person with speech difficulties and a condition regarding at least any one of an environment around the person with speech difficulties, weather in a location where the person with speech difficulties is present, and a body of the person with speech difficulties.

7. The inference model generation system according to claim 5, wherein the condition includes conditions regarding a motion of the person with speech difficulties, an environment around the person with speech difficulties, weather in a location where the person with speech difficulties is present, and a body of the person with speech difficulties.

8. The inference model generation system according to claim 3, wherein
the data set acquisition module acquires, as the data set, data indicating information on each of a plurality of items,
a weight coefficient for each of the plurality of items is determined according to each of the plurality of combinations, and
the inference model generation module generates the inference model for each of the plurality of combinations by using the weight coefficient according to the subject combination.

9. The inference model generation system according to claim 8, wherein the condition includes a condition regarding a motion of the person with speech difficulties and a condition regarding at least any one of an environment around the person with speech difficulties, weather in a location where the person with speech difficulties is present, and a body of the person with speech difficulties.

10. The inference model generation system according to claim 8, wherein the condition includes conditions regarding a motion of the person with speech difficulties, an environment around the person with speech difficulties, weather in a location where the person with speech difficulties is present, and a body of the person with speech difficulties.

11. The inference model generation system according to claim 2, wherein
a plurality of thought options is prepared in advance according to each of the plurality of combinations, and
the data set acquisition module acquires, as the data set for each of the plurality of combinations, data that indicates, as the thought, a thought option selected based on the reaction from among the plurality of thought options according to the subject combination by a person who cares for the person with speech difficulties.

12. The inference model generation system according to claim 11, wherein the condition includes a condition regarding a motion of the person with speech difficulties and a condition regarding at least any one of an environment around the person with speech difficulties, weather in a location where the person with speech difficulties is present, and a body of the person with speech difficulties.

13. The inference model generation system according to claim 11, wherein the condition includes conditions regarding a motion of the person with speech difficulties, an environment around the person with speech difficulties, weather in a location where the person with speech difficulties is present, and a body of the person with speech difficulties.

14. A thought inference device comprising:
an inference module configured to infer a second thought for a case where a second reaction is seen by inputting input data indicating a second condition for a case where the second reaction is seen to an inference model generated for a time frame and a location in which the second reaction is seen by the inference model generation system according to claim 2; and
an output module configured to output the second thought.

15. An inference model generation method comprising:
acquiring a plurality of data sets, each of the data sets indicating a condition for a case where a physical reaction is seen in a person with speech difficulties and a thought of the person with speech difficulties for a case where the physical reaction is seen; and
generating, for each of a plurality of combinations of a time frame and a location, an inference model by machine learning in which the condition indicated in the data set acquired in a time frame and a location of the subject combination is used as an explanatory variable and the thought indicated in the data set is used as an objective variable.

16. A non-transitory computer readable storage medium having stored thereon a computer program, the computer program letting a computer execute processing comprising:
acquiring a plurality of data sets, each of the data sets indicating a condition for a case where a physical reaction is seen in a person with speech difficulties and a thought of the person with speech difficulties for a case where the physical reaction is seen; and
generating, for each of a plurality of combinations of a time frame and a location, an inference model by machine learning in which the condition indicated in the data set acquired in a time frame and a location of the subject combination is used as an explanatory variable and the thought indicated in the data set is used as an objective variable.
